(19) United States
(12) Patent Application Publication
(10) Pub. No.: US 2005/0197647 A1
(43) Pub. Date: Sep. 8, 2005

(54) Surgical wound drain tube with flow control safeguards

(76) Inventors: Phillip B. Dolliver, Framingham, MA (US); Mark Edward Saunders, Attleboro, MA (US); Alec Bobroff, Norfolk, MA (US)

Correspondence Address: HAEMONETICS CORPORATION, 400 WOOD ROAD, BRAINTREE, MA, 02184-9114, US

(21) Appl. No.: 10/791,102

(22) Filed: Mar. 2, 2004

(51) Int. Cl. A61M 1/00; A61M 27/00

(52) U.S. Cl. 604/541

(57) ABSTRACT

Flow control safeguards are integrated into a blood collection system on a disposable component such as the post-operative surgical drain tube leading from the patient to the collection device. One aspect of the invention prevents suction levels that are too high for the tissue and organs adjacent at the surgical site by providing a vacuum relief valve integrated into the drain tube. In another aspect of the invention, reverse flow of fluid back to the patient is prevented by integration of an anti-reflux valve into the surgical drain tubing.

### FIELD OF THE INVENTION

The invention relates to devices used to collect blood and other fluids from a patient during and following a medical procedure. In particular, the invention relates to devices and methods for post-operative drainage of fluids from a surgical site.

### BACKGROUND OF THE INVENTION

Automated devices configured to collect blood and other fluids from a surgical site are common. In a post-operative setting, such devices may be configured as an autotransfusion system that returns collected blood back to the patient after processing and filtering. The collection devices operate by providing suction through a conduit such as tubing that is connected to a drain catheter placed at the surgical area. Blood and fluids are suctioned into the tubing and carried to a reservoir external to the patient.

Though collection of fluids from an internal surgical site is important to the recovery of the patient, there are risks involved with introducing suction to a surgical site. One danger to the patient is improper flow through the tubing placed at the surgical site. If suction is applied that is too strong, the treatment area, as well as organs and tissues adjacent to it, may be disturbed and negatively impacted. Another concern is reversal of flow back to the surgical site through the tubing if suction is discontinued. For example, in the absence of suction through a drain catheter placed in a chest cavity, normal breathing and movement of the diaphragm to expand the lungs will create vacuum in the cavity that could suction the contents of the catheter and tubing back to the surgical site. Flow through the tubing could impact a patient's breathing as suction in the chest cavity to inflate the lungs is reduced by the pressure leak represented by the catheter. Also, the re-introduction of possibly contaminated fluids and blood to the site could lead to infection or other complications.

The concerns discussed above are significant in a post-operative environment, because tissues and organs of the surgical site are weakened and more susceptible to damage by improper flow that may accidentally be introduced through the suction tubing of a blood collection device. Maintaining proper suction levels is important in post-operative collection devices, and especially for perioperative collection systems that are used during surgery and also postoperatively. Perioperative devices collect blood at higher suction levels during surgery then must transition to lower suction levels as their use continues during the post-operative period in order to reduce trauma to the surgical site. A risk exists that a perioperative device may accidentally be operated at high intraoperative suction levels that are too high for a post-operative condition, possibly damaging tissues or organs at the surgical site. The consequences of such an error would be especially damaging of occurring during use of a perioperative device in a procedure carried out in the chest cavity, such as a cardiac or spinal procedure, because of the proximity of critical organs such as the heart and lungs.

It would be desirable to provide flow control safe guards in the suction/fluid pathway of a blood collection device that would ensure that suction levels did not increase to an unsafe magnitude and that prevented positive reversal of flow through the collection tubing. It also would be desirable to provide such safeguards built into a component of the collection system that operators must use with each new procedure, such as part of a disposable set, so that the safeguards are certain to be in place regardless of the age and safety features of the permanent hardware and equipment. It is an object of the present invention to provide devices and methods for employing such flow control safeguards.

### SUMMARY OF THE INVENTION

The present invention provides a surgical drain tube having one or more components that serve to control the flow through the tube in a manner that is safe for the patient and non-disruptive to the post-operative surgical site. In particular, the drainage tube may be configured as a length of tubing configured to extend between a drainage catheter placed at the operative site in a patient and a blood collection device such as an autotransfusion device that can be operated to apply suction to the tubing to remove blood and other fluids from the site in order to promote healing. The tubing is configured with one or more flow control features built into it to ensure that flow occurs only in the correct direction and only at suction levels that are suitable for the surgical site.

In one aspect of the invention, the surgical drain tube may be configured to have a vacuum relief valve placed in fluid communication with the fluid/suction pathway defined by the lumen of the tubing in order to prevent suction in the line that is too great for the organs and tissues at the surgical site. The valve is configured to open automatically when vacuum levels in the fluid/suction pathway exceed a predetermined level above which tissues and organs could be disturbed. If a suction level that is greater than the pre-determined level is encountered in the lumen of the tubing, the valve will open automatically to a source of reduced suction, such as atmospheric pressure, and the suction force in the drain will be reduced to an acceptable level.

In another aspect of the invention an anti-reflux valve may be employed in-line in the drain tube. The anti-reflux valve is a check valve that permits fluid flow through the lumen of the tubing in only one direction. The valve is arranged to maintain flow away from the patient. The tubing can be further configured to have fail-safe connectors at its ends so that it can only be joined to the drainage catheter and collection device in an orientation that arranges the anti-reflux to maintain flow away from the patient.

With the anti-reflux valve in place, air and liquid being suctioned from the surgical site will not be free to flow backwards toward the patient if the suction is discontinued. In a procedure carried out in the chest cavity suction is generated as the diaphram moves to expand the lungs. A drain tube in place without such a valve provides an open fluid pathway into the chest cavity reducing suction needed to fill the lungs with air to carry out breathing. Additionally, reverse suction though the drain tube can be generated, drawing in blood and fluids previously drained from the surgical site. At a minimum such an occurrence hampers progress of cleaning the site from contaminants and blood from the surgery, but also fluid build up in the cavity could be harmful to adjacent organs such as the lungs.

The inventive drain tube may employ either one or both of the flow control safeguards discussed above.

It is the object of the present invention to provide safeguards for controlling flow through a surgical drain tube so that the patient is not injured by undesirable flow characteristics through the tube.

It is another object of the invention to provide a surgical drain tube having one or more flow control devices that insure flow characteristics through the tube that are not deleterious to the condition of the treatment site and surrounding area.

It is another object of the invention to provide a surgical drain tube having an in-line anti-reflux valve to ensure fluid through the tube flows only away from the patient.

It is another object of the invention to provide a surgical drain tube having a suction relief valve in fluid communication with the lumen of the drain tube and that automatically vents to a source of reduced suction or to atmospheric pressure when suction levels in the drain tube exceed a predetermined threshold.

It is another object of the invention to provide a surgical drain tube having an in-line anti-reflux valve and integral suction relief valve to ensure that flow in the drain tube is maintained in the proper direction and at appropriate levels for the patient.

It is another object of the invention to provide a surgical drain tube that is compatible for use with a variety of blood collection devices but especially with perioperative autotransfusion systems including systems intended for use in the chest cavity for cardiac procedures.

It is another object of the invention to provide a method of controlling of the flow through a surgical drain tube that comprises integrating one or more flow control safeguards.

### BRIEF DESCRIPTION OF THE DRAWINGS

The foregoing and other objects and advantages will be appreciated more fully from the following further description thereof, with reference to the accompanying diagramatic drawings wherein:

FIG. 1 is a diagramatic illustration of the surgical drain tube of the present invention in use, joined to a patient and to a blood collection device;

FIG. 2 is a side sectional view of a surgical drain tube employing an anti-reflux valve and a suction relief valve;

FIG. 3A is a side view of a surgical drain tube employing an anti reflux and a suction relief valve joined to an autotransfusion device collection vessel;

FIG. 3B is a diagrammatic illustration of an variable volume rotor autotransfusion device in fluid communication with the drain tube of the present invention;

FIG. 4A is a detailed side sectional view of an anti-reflux valve in the closed configuration;

FIG. 4B is a detailed side sectional view of an anti-reflux valve in the open configuration;

FIG. 5 is a detailed side sectional view of a vacuum relief valve in the closed configuration.

### DESCRIPTION OF THE ILLUSTRATIVE EMBODIMENTS

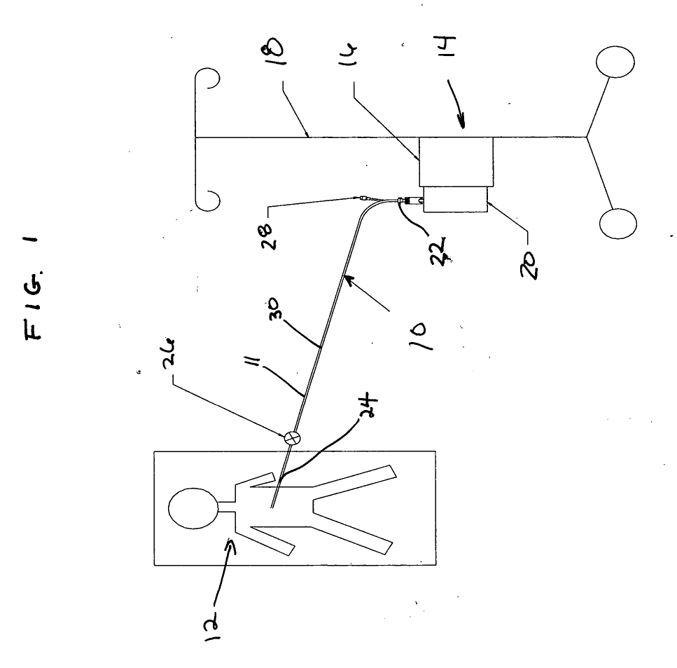

FIG. 1 is a diagrammatic illustration of a surgical drain tube 10 of the present invention extending between the patient 12 and a blood collection device 14. The drain tube may be used with a compact perioperative autotransfusion device 16 such as the OrthoPAT® manufactured by Haemonetics® Corporation and described in U.S. Pat. No. 5,733,253, the entirety of which is incorporated by reference herein. The autotransfusion device may be sufficiently compact to be mounted on an IV pole 18 which is readily available in a hospital setting. The blood collection device 14 is operated to create suction in a fluid collection vessel 20 to which a device end 22 of the drain tube is joined. A patient end 24 of the drain tube is configured to be joined to a drainage catheter in place in the surgical site of the patient 12. Drain tube 10 has one or more flow control safeguards such as an anti-reflux 26 and/or a vacuum relief valve 28 that are in fluid communication with lumen 30 that extends through tubing 11 that defines drain tube 10.

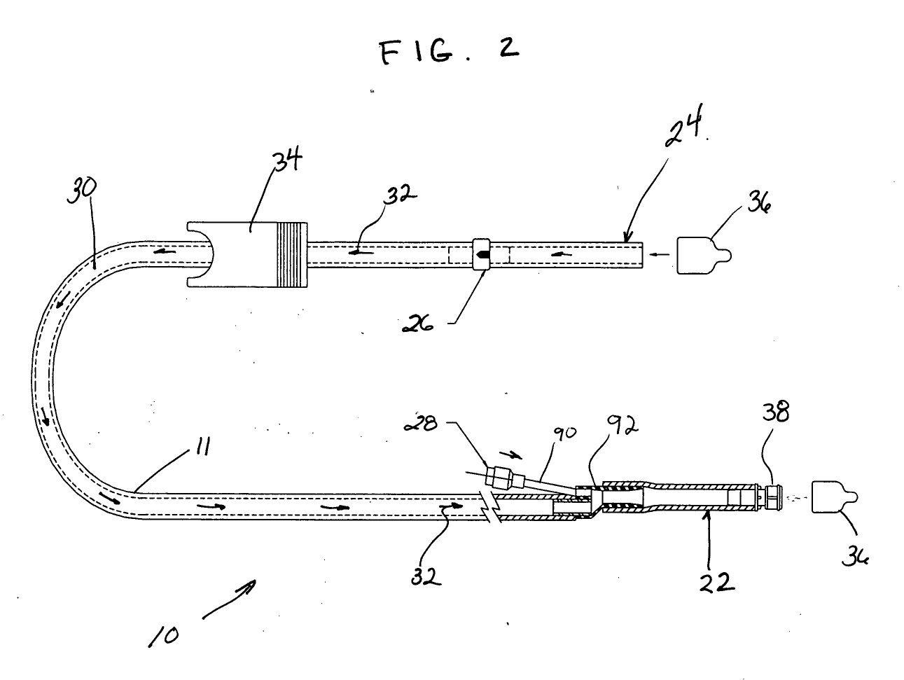

FIG. 2 shows a side sectional view of the surgical drain tube 10 according to present invention that employs both an anti-reflux valve 26 and vacuum relief valve 28. The drainage tube 10 is formed from a length of tubing 11 having at least one lumen 30 throughout its length through which air, blood or other bodily fluids may pass. The lumen 30 defines a pathway through which suction is applied and a pathway through which fluid collected by the suction is carried. The flow control safeguards may comprise an anti-reflux valve 26 and/or a suction relief valve 28 and other electro mechanical controls. To keep the controls simple, economical to produce and easily integrated into a single drainage tube that is a disposable product, it is beneficial to have controls that can be joined in-line in the tubing. To sense and control flow in the drain tube the flow controls are positioned to be in fluid communication with the lumen 30, which defines a suction/fluid pathway of the drain tube.

The anti-reflux valve 26 ensures that the flow occurs only in the direction indicated by arrows 32 shown in FIG. 2. The arrows 32 represent flow away from the patient and to the collection device. The anti-reflux valve is configured to close the passageway through the lumen 30 if flow of fluid occurs in the opposite direction through the tubing 11. If the suction pressure increases to a magnitude that is unsafe, relief valve 28 will be pulled open by the suction force to open the fluid passageway to a reservoir of reduced suction pressure or to the ambient pressure to reduce the magnitude suction in the tubing 11.

An external manual ratchet clamp 34 may be provided with the drain tube 10 to provide an operator with the capability of manually preventing flow through the tube during its set up with a drain catheter and blood collection device device. Also, end caps 36 may be provided over the ends of tube 10 to prevent contamination during shipping and storage and are removed just prior to use. In connecting the tube 10 to the drain catheter and auto transfusion device a fail-safe connection fitting 38 ensures the operator does not connect the tubing in reverse. A fail-safe fitting 38 may be integrated either at the device end 22 or patient end 24 of the tube and is configured to mate only with a compatible fitting located on the end of the drainage catheter or on the collection device. In the example shown in FIG. 2, the fitting is shown joined to the device end 22 of the tube. The fail-safe feature ensures that the anti-reflux valve is oriented in the correct direction during use.

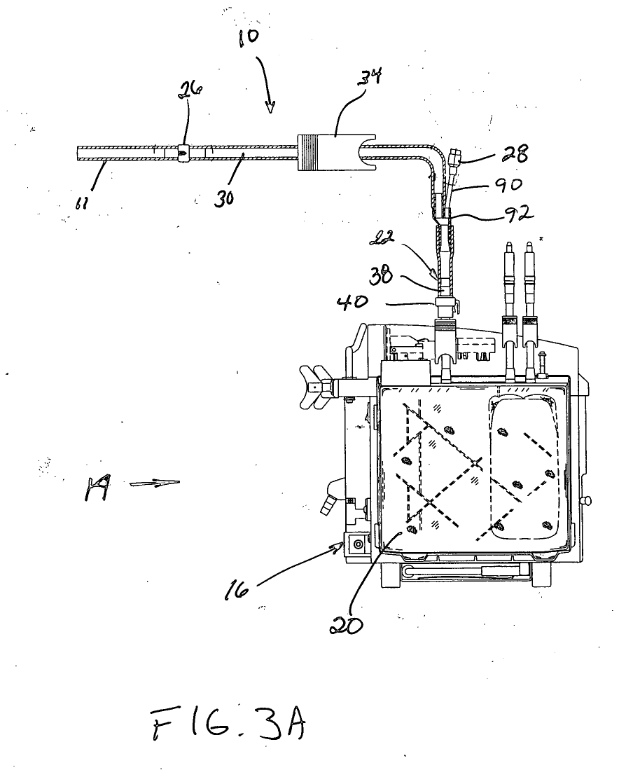

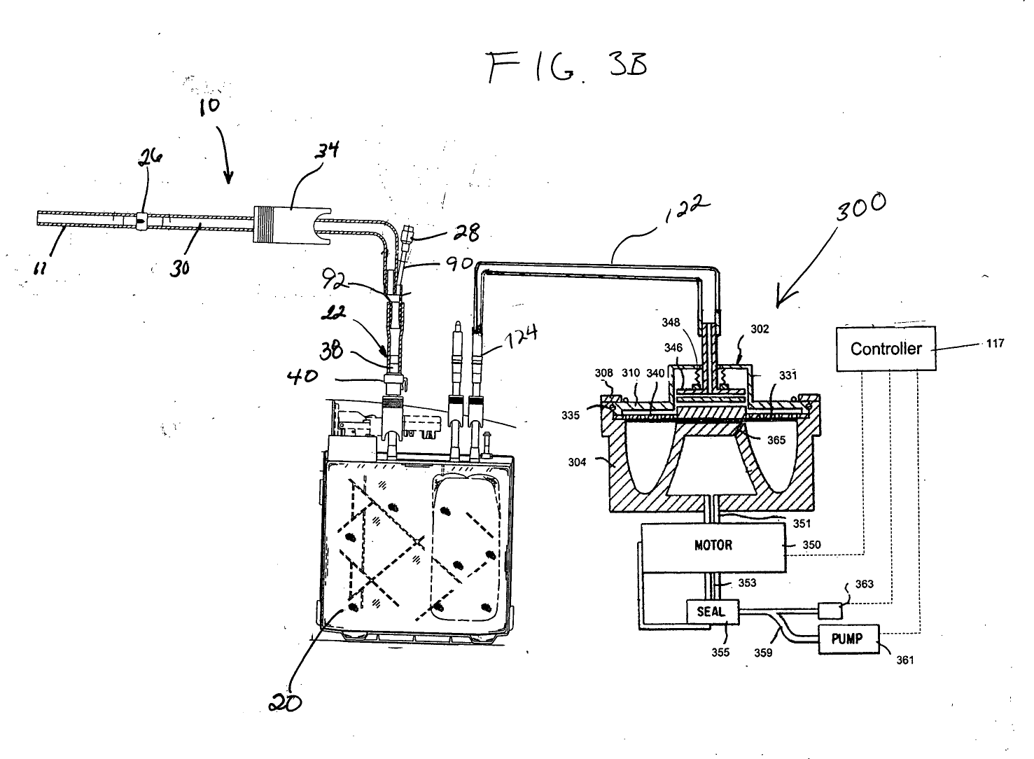

FIG. 3A shows an illustration of the drain tube 10 connected at its device end 22 to an autotransfusion device 16. Tube 10 can be configured to attach to any autotransfusion device, a fail-safe connector 38 at the device end of the tube mates and securely engages a compatible fail-safe connector 40 at the opening to the fluid/suction pathway of the device 16. As shown in FIG. 3, the fluid/suction pathway of the device in this example begins with a collection vessel 20 into which the fluid suctioned from the patient is collected prior to further processing by the autotransfusion device or other equipment.

The collection device 14, such as autotransfusion device 16, has a pump mechanism that creates suction in the fluid/suction pathway to which the tube 10 is connected. The device 16 may be a perioperative device that can be used during both intraoperative and post operative site drainage. Because a perioperative device must operate at higher suction levels during an operation, then operate at reduced suction levels in the postoperative environment to avoid damage to the tissues at the now closed surgical site, the tube of the present invention offers an important benefit. When the post operative drain tube of the present invention is installed to the drain catheter and to the device, the danger of operating the device at the higher suction levels intended for an intraoperative environment is eliminated because the vacuum relief valve 28 will instantly and automatically relieve the suction levels to avoid injury if suction is too great. This flow control safety feature is especially important in procedures carried out in the chest cavity such as cardiac or spine procedures where suction is likely to affect vital organs such as the lungs and heart.

An example of a perioperative autotransfusion device is the OrthoPAT® manufactured by Haemonetics Corporation and described in U.S. Pat. No. 5,733,253. The OrthoPAT®) device 16 is shown as the collection device 14 in the example illustrated in FIG. 3A. The OrthoPAT® is used in orthopedic procedures but a similar device can be configured to operate at lower pressures as required in cardiac procedures as well.

FIG. 3B illustrates portions of an OrthoPAT autotransfusion device 16 in more detail. The variable-volume rotor 302 illustrated is of a type described in U.S. Pat. No. 5,733,253 at FIGS. 1-4, although other rotors shown and described in this patent may be used as well, such as the rotors shown in FIG. 7, 8A, or 41 and 42 of that patent. The variable-volume rotor 302 has an elastic diaphragm 331 and a rigid member 310, which together define a chamber of varying volume, as described in U.S. Pat. No. 5,733,253. The rotor is in fluid communication with drain tube 10 and collection vessel 20 via rotor tube 122 that is connected to tank fitting 124, which opens to the interior of the collection vessel. Fluid communication in and out of the rotor is provided by a collector assembly 346 which is attached to tube 122 and is connected to the rigid member 310 via a rotary seal 348. The tubing 122 and the collector assembly 346 are held stationary while the rest of the variable-volume rotor 302 rotates (i.e., the rigid wall 310 and the diaphragm 331). To protect the elastic diaphragm 331 while spinning from the stationary collector assembly 346, a perforate interior wall 340 is attached below the rigid wall 310. The perforate interior wall 340 includes holes that allow fluid communication between the areas of the chamber above and below the perforate interior wall 340.

In use, the variable volume rotor 302 is held onto and spun by a centrifuge chuck 304. The chuck 304 holds the rotor 302; the chuck 304 has a clamp 308 that holds the rotor 302 securely in place in the chuck 304, and an O-ring 335 that forms an air-tight seal. A drive motor 350 is connected to the chuck 304 by means of a shaft 351. In order to apply a vacuum or pressure to the rotor 302 to pump fluid in and out of the rotor, respectively, the shaft 351 has an axial hole through its center 353 and is connected to a rotary pneumatic seal 355, which in turn is connected by tubing 359 to a compressor/vacuum pump 361 and to a controllable exhaust valve 363. Holes 365 in the interior of the chuck 304 allow air to flow to and from the compressor/vacuum pump 361. These spinning and pumping mechanisms are controlled by a controller 117.

To draw blood from the wound, controller 117 controls the compressor/vacuum pump 361 to provide a vacuum through the chuck to the exterior side of the diaphragm 331. Because the diaphragm 331 is pulled downward by the vacuum in the chuck 304, an area of low pressure is created in the chamber, causing suction to be applied at the drain catheter 102. Consequently, fluid is drawn into the rotor 302 through the rotor tube 122. As more and more fluid enters the rotor 302, the diaphragm 331 changes shape to accommodate it. In this manner, blood and/or other fluid is drawn from the wound-drain site through the drain catheter 112 and associated tubing 122/valving 149 into the rotor 302.

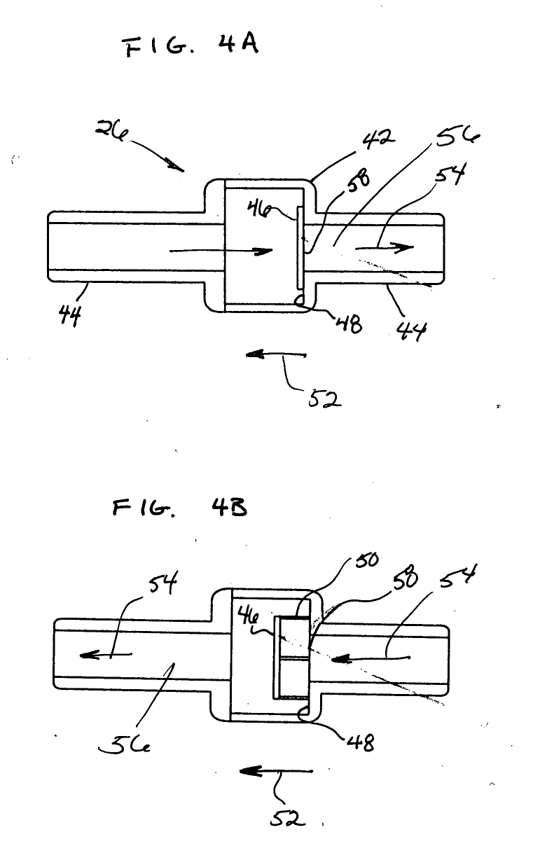

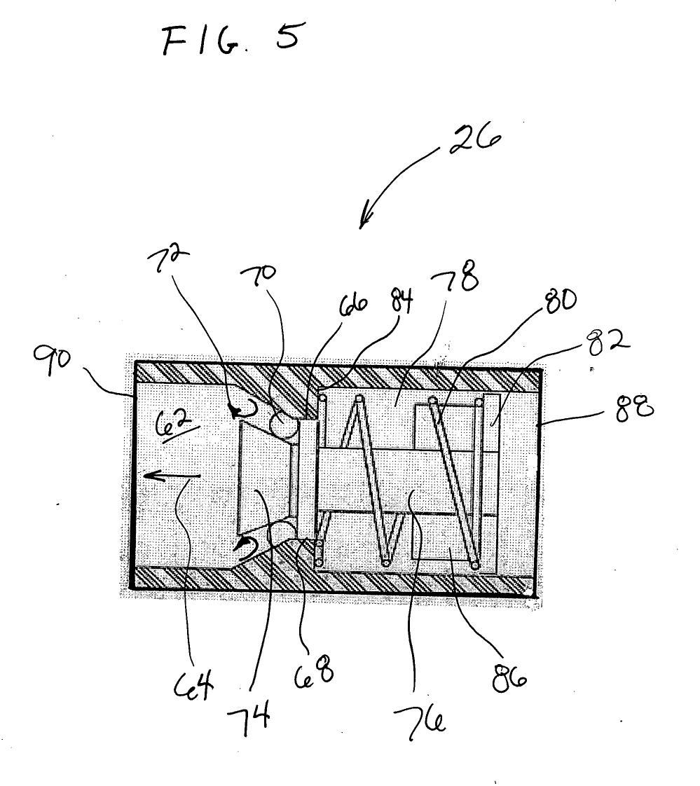

FIG. 4A shows a detailed sectional side view of an illustrative configuration of the anti-reflux valve 26. The anti-reflux valve may comprise a rigid body 42 having a nipple 44 extending from each end over which is received segments of tubing 11 such that the nipples are received into the lumen 30 of the tubing. When assembled with tubing segments on the nipples 44, the resultant assembly is an integral tube 10 having built-in anti reflux valve.

In the body of the valve 26 is a flexible parachute valve element 46 joined to a patient side interior wall 48 by a plurality of flexible legs 50. The valve element may be integrally formed with the legs from silicon material. Such a valve is commercially available from Resenex Corporation of Chatsworth Calif. under part number R-702HF.

In FIGS. 4A and 4B, arrow 52 indicates the direction of intended flow through the valve 26. Arrows 54 indicate the direction of suction actually experienced in the fluid pathway 56 of the valve. It is intended that the direction of suction in flow through the valve only be away from the patient and to the device (leftward in the figures) rather than toward the patient. In FIG. 4B, the auto transfusion device is operating normally and suction, as indicated by arrows 54, is being directed safely away from the patient and toward the device. Suction in the direction away from the patient (leftward) pulls the parachute element 46 away from the patient a side interior wall 48 of the valve body 42, keeping the element 46 tethered by the legs 50 away from the valve opening 58 so that fluid may pass freely through the valve.

In FIG. 4A the direction of suction is toward the patient as indicated by arrows 54 (rightward in the figure). Such a condition may arise if the collection device stops operating or if the tube 10 became disconnected from the device. When the device stops operating, it may be configured to automatically vent the drain tube to atmospheric pressure. In either event, the discontinuation of suction from the device 14 prevents internal suction from the patient to reverse flow through the tube back to the patient. In the case of procedures carried out in the chest cavity, such as cardiac procedures, when the patient breathes and diaphragm expands, vacuum is created in the chest cavity to fill the lungs. However, the suction created in the chest cavity also redirects suction in the drain tube back toward the patient as reflected by the arrows 54 in FIG. 4A. Suction experienced in this direction causes the parachute valve 46 to move rightward and seal shut against the valve opening 58 to prevent flow in the direction of the patient. The valve element 46 quickly responds to the change in the direction of the suction due to its flexibility and the flexibility of the legs 50 that are configured to extend and collapse flat around the circumference of the valve element.

FIG. 5 shows a sectional side view of a vacuum relief valve 26 that may be employed in the tube of the present invention. The valve is shown in the closed configuration. Suction chamber 62 is in fluid communication with the lumen 30 of the tube 10. Arrow 64 indicates the direction of suction that is applied in the chamber 62 when the tube is in use. Valve opening 66 is closed by piston flange 68 and sealed by O-ring 70 under normal suction levels. If suction levels increase beyond a predetermined magnitude considered to be unsafe for the closed surgical site, suction currents indicated by arrows 72 act on the surfaces of frusto-conical element 74 to pull attached piston 76 leftward, in the direction of the suction to move the flange 68 from opening 66 to allow venting from the vent cavity 78.

The suction force acting on frusto-conical element 74 that is necessary to move the piston 76 and to open the valve is predetermined by the configuration of biasing spring 80. The biasing spring extends around the piston 76 in the vent chamber 78 and is compressed between valve wall 84 and spring flange 82 located at the end of piston 76. Spring guide 86 formed on the inner face of the spring flange helps to keep the spring concentric relative to the piston. The spring is biased in its expanded configuration to push spring flange 82 away from valve wall 84 so that piston flange 68 normally closes opening 66. However, the spring is configured to have a spring force that permits the spring to compress when the suction force experienced in the suction chamber reaches a predetermined level. When the predetermined suction level is reached, the spring begins to compress under the suction forces applied to frusto-conical element and the piston moves in the direction of the suction force (leftward) to open valve. For procedures carried out in the chest cavity, spring is calibrated to open the valve when approximately 70 cm H₂O of vacuum is reached in the suction pathway of the tube 10 and suction chamber 62 of the valve.

As the valve starts to open, the suction flow enters the vent chamber 78 through the opening 66. Suction pulls on the spring flange 82 of piston 76 to further open the valve. Vent cavity port 88 is open to ambient (atmospheric) pressure which effectively vents the tube of the excess suction. Alternatively, the vent cavity could be in communication with a closed environment that is at a reduced suction level or at positive pressure in order to reduce the amount of vacuum in the suction pathway of the tube 10 when valve 26 is opened. A vacuum relief valve such as that discussed above is commercially available from Smart Products of San Jose, Calif. under part no. 130 CYB-1#.

As best seen in FIGS. 2-3A, the vacuum relief valve may be secured in fluid communication with the suction pathway defined by the lumen 30 via an extension tube 90 secured into a Y-connector fitting 92 that is in-line with tubing 11. With the base of the Y-connector and one leg joined to sections of the tubing 11, a third leg is left available for receiving the vacuum relief valve. However, it should be appreciated that use of the Y-connector is but one convenient way to integrate the vacuum relief valve into the suction pathway of the tube 10. Other arrangements or configurations of fittings can be used and are considered to be within the scope of the invention.

In use, the surgical drain tube is provided to the user as an assembled, one-piece disposable component integrating one or more of the flow control safeguards discussed above. After a surgical procedure, one or more drain catheters have already been placed at the surgical site and the site closed. The end of the drain catheter is left available outside the patient for connection to a post-operative surgical drain tube. The user removes a cap 36 from the patient end 24 of the tube and attaches it to the end of the drain catheter using a ribbed inner sleeve insertable into the lumen of the catheter and into the lumen of the drain tube. If equipped with a fail-safe connector at the device end 22, the user will be unable to connect the wrong end of the drain tube to the drain catheter, and correct orientation of the tube to obtain proper flow through the anti-reflux valve will be ensured.

Next, the user removes the cap 36 from the device end 22 of the tube and secures the end to the collection device 14. If fail-safe connections 38 and 40 are employed, the connection may be a snap fit or quick-release clamp or the like employing a tongue on one fitting and mating groove on the other.

The operator then may start the collection device to begin suction through the system. If the suction is inadvertently set too high for a post-operative environment, the vacuum relief valve 26 will open automatically to vent the tube 10 to a source of higher pressure. If suction is discontinued, for example by powering off the collection device, the anti-reflux valve prevents reverse flow of fluid through the tube back to the patient.

Accordingly, a surgical drain tube with flow control safeguards has been described. The tube is easy to use in a postoperative wound drain procedure. Because it integrates safeguard features all in one disposable tubing set, the safety benefits can be enjoyed with existing equipment and cannot be inadvertently by-passed, ensuring a safer procedure for the patient.

It should be understood, however, that the foregoing description of the invention is intended merely to be illustrative thereof and that other modifications, embodiments and equivalents may be apparent to those skilled in the art without departing from its spirit. Having thus described the invention what we desire to claim and secure by letters patent is:

1. A surgical drain tube with flow control safeguards comprising:
a length of tubing having at least one lumen extending therethrough and having a patient end configured to be joined to a wound drain catheter and a device end configured to be connected to a collection device and;
a vacuum relief valve having one end in fluid communication with the lumen of the tubing and one end in fluid communication with a fluid source.

2. A surgical drain tube as defined in claim 1 wherein:
the vacuum relief valve is configured to open when a predetermined level of vacuum is reached in the lumen of the drain tube.

3. A surgical drain tube as defined in claim 2 wherein:
the vacuum relief valve is configured to open when vacuum in the lumen of the tubing is approximately seventy (70) centimeters of H₂O.

4. A surgical drain tube as defined in claim 2 wherein:
the vacuum relief valve further comprises a piston for closing and opening an orifice and the piston is biased in the closed position by a spring calibrated to permit opening at the pre-determined vacuum level.

5. A surgical drain tube as defined in claim 1 wherein:
the end of the vacuum relief valve open to a source of fluid is open to atmospheric pressure.

6. A surgical drain tube with flow control safeguards comprising:
a length of tubing having at least one lumen extending therethrough and having a patient end configured to be joined to a wound drain catheter and a device end configured to be connected to a collection device and;
an inline anti-reflux valve configured as a check valve to ensure fluid flow through the lumen of the tube in only one direction.

7. A surgical drain tube as defined in claim 6 wherein the anti-reflux valve is configured to permit fluid flow only away from the patient end of the tubing and prevent flow back to the patient.

8. A surgical drain tube with flow control safeguards comprising:
a length of tubing having at least one lumen extending therethrough and a patient end configured to join to a drainage catheter and a device end configured to join to a perioperative autotransfusion system;
a vacuum relief valve joined to the tubing and having one end in fluid communication with the lumen and;
an inline anti-reflux valve configured to permit fluid flow through the lumen only in the direction leading away to the patient.

9. A surgical drain tube as defined in claim 8 wherein the patient end of the tubing is securable only to the drainage catheter and the device end of the tubing is securable only to the autotransfusion device such that the ends cannot be inadvertently connected in reverse.

10. A method of controlling flow through a surgical drain tube in a post-operative environment comprising:
providing a drain tube having a lumen extending therethrough, a patient end configured to be joined to a drainage catheter, a device end configured to be joined to a collection device, and an inline anti-reflux valve configured to permit fluid flow through the lumen only in a direction leading away from the patient;
securing the patient end of the tube to a drainage catheter placed in a surgical site of the patient and joining the device end of the tube to a collection device and;
operating the collection device to apply suction through the lumen of the tube to cause flow from the patient to the collection device.

11. A method of controlling flow through a surgical drain tube as defined in claim 10 wherein the tube further comprises a vacuum relief valve having one end in fluid communication with the lumen of the tubing and another end in fluid communication with a source of fluid and being configured to open to the source of fluid when a pre-determined vacuum level is reached in the lumen of the tube.
